(19) United States
(12) Patent Application Publication
(10) Pub. No.: US 2008/0113402 A1
(43) Pub. Date: May 15, 2008

(54) Magnetic Particle Capture of Whole Intact Organisms from Clinical Samples

(75) Inventor: Ray McMillian, Timonium, MD (US)

Correspondence Address: David W. Highet;Becton Dickinson and Company, 1 Becton Drive, MC110, Franklin Lakes, NJ, 07417-1880, US

(73) Assignee: BECTON DICKINSON AND COMPANY, Franklin Lakes, NJ (US)

(21) Appl. No.: 12/015,719

(22) Filed: Jan. 17, 2008

Related U.S. Application Data
(63) Continuation of application No. 10/902,871, filed on Aug. 2, 2004.

(51) Int. Cl. C12Q 1/02

(52) U.S. Cl. 435/29; 210/695; 435/173.1; 435/173.9; 435/176; 435/201

(57) ABSTRACT

A method includes providing a sample containing at least one whole, intact particle or organism in a container; creating a mixture comprising the sample, at least one magnetically-responsive particle, and a remainder; and providing the mixture with a pH of less than about 7.0. By providing the mixture with a pH of less than about 7.0, alteration of the surface charge properties of at least the one magnetic particle occurs, thereby causing the at least one whole, intact particle or organism to become non-specifically bound to the at least one magnetically-responsive particle to form a complex.

### FIELD OF THE INVENTION

The present invention is directed to compositions and methods for extracting, concentrating and/or isolating whole, intact particles or organisms from a sample. More particularly, the present invention is directed to compositions and methods for extracting, concentrating and/or isolating whole, intact particles or organisms from samples via reversible binding with magnetically-responsive particles.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The isolation and/or separation of biological components from a sample is a necessary task in many diagnostic and biochemical procedures. Known techniques for accomplishing this objective include lysing of biological materials to release the nucleic acids contained therein, followed by separation of at least a portion of the nucleic acid. The nucleic acid can be separated and/or removed via a number of different techniques. One such technique involves reversibly binding the nucleic acid to magnetic particles. Such techniques are described in U.S. Pat. Nos. 5,973,138 and 6,433,160, the contents of which are incorporated herein by reference in their entirety. It is desirable, however, to concentrate, isolate or remove whole, intact particles or organisms from a sample.

### SUMMARY OF THE INVENTION

The present invention is directed to compositions and techniques that non-specifically associate whole, intact particles or organisms with magnetic particles by altering the surface charge characteristics of the magnetic particles and/or the surface charge characteristics of the particles or organisms themselves. Thus, the whole, intact particles or organisms are non-specifically associated with the magnetic particles without precipitation of these particles or organisms out of solution.

According to one aspect, the present invention provides a method comprising providing a sample containing at least one whole, intact particle or organism, creating a mixture, that comprises the sample, at least one magnetically-responsive particle, and a remainder, and providing the mixture with a pH of less than about 7.0. By providing the mixture with a pH of less than about 7.0, alteration of surface charge properties of at least the one magnetically-responsive particle occurs, thereby causing the at least one whole, intact particle or organism to become non-specifically bound to the at least one magnetically-responsive particle to form a complex.

### BRIEF DESCRIPTION OF THE DRAWING

The foregoing and other features, aspects and advantages of the present invention will become apparent from the following description, appended claims and the exemplary embodiments shown in the drawing, which is briefly described below. It should be noted that, unless otherwise specified, like elements have the same reference numbers.

FIG. 1 is a schematic illustration of an embodiment of a process performed according to the principles of the present invention.

### DETAILED DESCRIPTION OF THE INVENTION

The principles of the present invention will now be further described by the following discussion of certain illustrative embodiments thereof and by reference to the foregoing drawing FIGURE.

As used herein, “whole, intact particles or organism” means any naturally occurring or synthetic modification of a whole particle or organism that has not been lysed or otherwise broken down into constituent components. Whole, intact particles or organisms include, but are not limited to, whole cells, bacteria, viruses, parasites and combinations of the foregoing.

As used herein, “sample” means any biological particle or organism-containing substance including, but not limited to, blood, plasma, serum, urine, bone marrow aspirates, cerebral spinal fluid, tissue, cells, food, feces, saliva, oral secretions, nasal secretions, bronchial lavage, cervical fluids and lymphatic fluids. Optionally, the sample may be sterile.

As used herein, “magnetically-responsive particle” means a particle is capable of having a magnetic moment imparted thereto or otherwise moveable under the action of a magnetic field.

As used herein, “non-specifically bound” means the binding mechanism does not occur via a receptor, capture agent, or the like, which would selectively couple with a specific agent.

The Applicant has found that when in an acidic environment, magnetically-responsive particles will reversibly bind to whole, intact particles or organisms. Although not desiring to be bound by a particular theory, the Applicant believes that an acidic environment increases the electropositive nature of the particles, thereby increasing the binding of the particles to the electronegative whole, intact particles or organisms.

According to a preferred embodiment of the present invention, the magnetically-responsive particles are preferably uncoated or otherwise untreated. Thus, the particles bind non-specifically to the whole, intact particles or organisms. Particles useful in the present invention include iron particles, and the iron may be an iron oxide of forms such as ferric hydroxide and ferrosoferric oxide, which have low solubility in an aqueous environment. Other iron particles such as iron sulfide and iron chloride may also be suitable for binding and extracting nucleic acids using the conditions described herein.

The shape of the magnetically-responsive particles is not critical to the present invention. The magnetically-responsive particles may be of various shapes including, for example, spheres, cubes, oval, capsule-shaped, tablet-shaped, nondescript random shapes, etc., and may be of uniform shape or non-uniform shapes. Whatever the shape of a particle, its diameter at its widest point is generally in the range of from about 0.1 μm to about 20 μm. According to one embodiment, the magnetically-responsive particles have a diameter of about 1.0 μm.

The acidic environment in which the magnetically-responsive particles effectively and reversibly bind whole, intact particles or organisms can be provided through a variety of means. For example, the magnetically-responsive particles can be added to an acidic solution, or an acidic solution may be added to the particles. Alternatively, a solution or environment in which the magnetically-responsive particles are located can be acidified by addition of an acidifying agent such as hydrochloric acid, sulfuric acid, acetic acid or citric acid. Provided that the environment in which the magnetically-responsive particles are located is of a pH less than about 7.0, the particles will reversibly bind whole, intact particles or organisms. According to a preferred embodiment, a pH of about 4.5-5.5 is established to promote binding.

One or more washing steps may optionally be performed at this stage to further eliminate undesirable substances. Any suitable wash may be utilized. For example, a non-ionic detergent or a non-ionic detergent/low concentration acid solution may be utilized.

The bound whole, intact particles or organisms can be eluted into an appropriate buffer for further manipulation. Heating the environment of the particles with bound whole, intact particles or organisms and/or raising the pH of such environment can accomplish such elution. Agents that can be used to aid the elution of whole, intact particles or organisms from magnetically-responsive particles include basic solutions such as potassium hydroxide, sodium hydroxide or any compound that will increase the pH of the environment to an extent sufficient that electronegative whole, intact particles or organisms are displaced from the magnetically-responsive particles. According to a preferred embodiment, a pH of about 8.3-8.4 is established to promote release of the bound particles or organisms.

The whole, intact particles or organisms can then be extracted, concentrated and/or isolated. Subsequently, the whole, intact particles or organisms can be subjected to further processes, such as one or more of the following: cultivation, polymerase chain amplification, strand displacement amplification, reverse transcriptase strand displacement amplification, and ligase chain amplification.

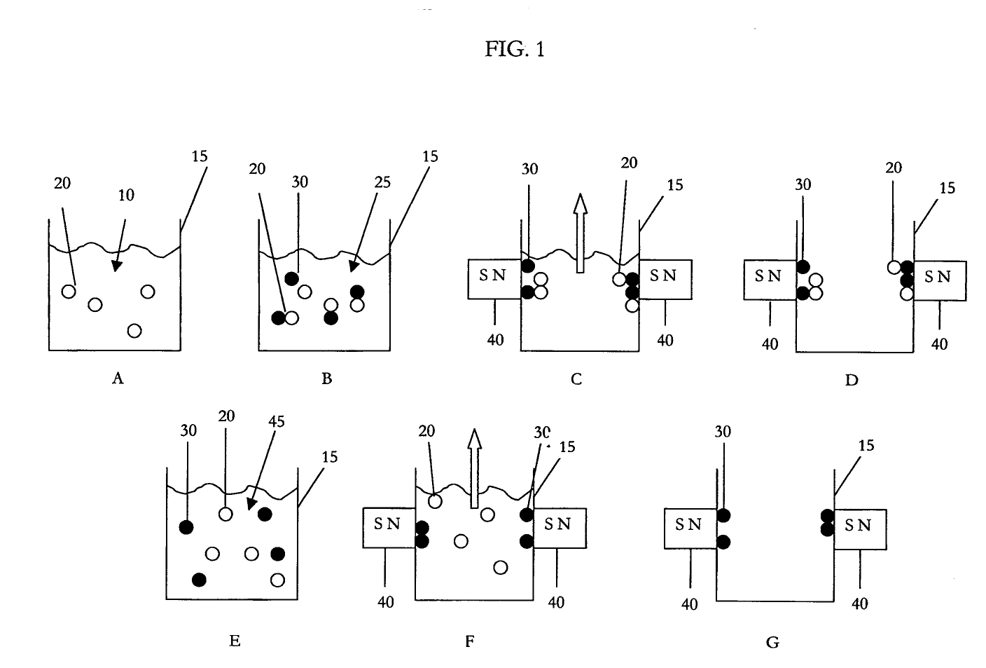

An exemplary process performed according to the principles of the present invention will now be described by reference to FIG. 1.

In step A, a sample 10 is located in a container 15. The sample 10 contains whole, intact particles or organisms 20.

A mixture 25 is then formed in step B that includes the sample 10, whole, intact particles or organisms 20 and magnetically-responsive particles 30. The pH of this mixture is brought to an appropriate level, preferably below about 7.0, more preferably about 4.5-5.5. The mixture can be formed by any suitable means. For example, the magnetically-responsive particles 30 can be added to an acidic solution, or an acidic solution may be added to the particles 30. Alternatively, a solution or environment in which the magnetically-responsive particles 30 are located can be acidified by addition of an acidifying agent such as hydrochloric acid, sulfuric acid, acetic acid or citric acid.

As previously described, the change in pH causes a modification of the surface charge characteristics of at least the magnetically-responsive particles 30, causing the whole, intact particles or organisms 20 to become bound to the magnetically-responsive particles 30, thereby forming a complex. A magnetic field is then applied. As illustrated in step C, this can be accomplished by bringing opposing permanent magnets 40, or electromagnets (not shown), into close proximity with the outside of the container 15. Under the influence of the magnetic field, the bound whole, intact particle or organism magnetically-responsive particle complex is drawn toward the magnets. The supernatant, or remainder, of the mixture 25 can them be removed from the container 15 (step D).

One or more washing steps (not shown) may optionally be performed at this stage to further eliminate undesirable substances. Any suitable wash may be utilized. For example, a non-ionic detergent or a non-ionic detergent/low concentration acid solution may be utilized.

Step E is illustrative of eluting the complex to free the whole intact particles or organisms 20 from the magnetically-responsive particles 30. This elution can be accomplished by any suitable means such as by chemical agent, thermal energy or a combination of the two. For example, a buffer agent 45 can be added to increase the pH to a suitable level. According to one embodiment, the pH is raised to approximately 8.3-8.4. The buffer may comprise KOH.

The magnets 40 are then brought back into close proximity with the container 15 in step F, which now draws just the magnetically-responsive particles 30 to the sidewalls of the container 15. The whole, intact particles or organisms 20 can then be removed from the container 15 (step G).

Subsequent to step G, the whole, intact particles or organisms 20 can be subjected to further processes, such as one or more of the following: cultivation, polymerase chain amplification, strand displacement amplification, reverse transcriptase strand displacement amplification, and ligase chain amplification.

The above-described steps of the exemplary process may be carried out manually, in automated fashion or by a combination of manual and automated steps. The automated steps may be performed with an automated robotic device, which optionally includes automated pipetting, mixing, and magnet positioning functionality. The automated robotic device may be computer controlled.

The present invention can be used in a number of different contexts. For example, the present invention may be utilized in connection with systems and methods of the type described in U.S. Pat. No. 6,672,458, the content of which is incorporated herein by reference in its entirety.

The principles if the present invention will now be describe by reference to the following illustrative, non-limiting examples.

### EXAMPLE 1

An experiment was performed to determine whether Staphylococcus aureus (S. aureus) and Escherichia coli (E. coli) could be extracted from an acidic buffer environment. The recovery of the microorganisms from the buffer was evaluated by examination of cultures prepared as described below.

A 1.0 ml quantity of 0.1 M sodium acetate buffer having a pH of 4.8 was pipetted into 2.0 ml microcentrifuge tubes, each tube containing 50 mg of ferrosferric oxide having an average particle size of approximately 1.4 microns. A 0.01 ml quantity of a S. aureas ATCC 25923 suspension at 1×10⁶ CFU/ml was added to one tube, and a 0.01 ml quantity of E. coli ATCC 11775 suspension at 1×10⁶ CFU/ml was added to a second tube.

The tubes containing the above-described mixture were rotated on a Nutator mixer for three hours at ambient temperature to promote binding of the iron oxide with the S. aureus and E. coli microorganisms. A neodymium magnet was then placed at the sides of the tubes for 30 seconds.

The supernatant was then removed from the tubes with a pipette. Some of the removed supernatant was used to make a 10-fold dilution. Both the undiluted and the diluted supernatant were applied to growth plates as described in more detail below.

The iron oxide/microorganism complex in the microtube was then washed twice with the above-mentioned sodium acetate buffer. The complex was then resuspended with 1 ml of the sodium acetate buffer. A portion of the suspension was then used to prepare a 10-fold dilution. Both the undiluted and the diluted suspension were applied to growth plates as described in more detail below.

A 0.1 ml quantity of each of the following samples were pipetted and spread onto each one of 3 different BBL™ blood agar plates (TSA II with 5% sheep's blood):
(i) undiluted S. aureus supernatant;
(ii) diluted S. aureus supernatant;
(iii) undiluted S. aureus iron oxide suspension;
(iv) diluted S. aureus iron oxide suspension;
(v) undiluted E. coli supernatant;
(vi) diluted E. coli supernatant;
(vii) undiluted E. coli iron oxide suspension; and
(viii) diluted E. coli iron oxide suspension.

The plates were incubated at 36° C. in ambient air for 24 hours. To determine the total recovery, the number of colonies were counted on each plate and multiplied by 10 for the undiluted sample, and multiplied by 100 for the diluted sample. The number of colonies calculated are reported in Tables I and II below.

TABLE I
S. aureus recovery

| Sample | Plate 1 | Plate 2 | Plate 3 | Average |
|---|---|---|---|---|
| (i) | 0 | 0 | 0 | 0 |
| (ii) | 0 | 0 | 0 | 0 |
| (iii) | TNTC* | TNTC | TNTC | TNTC |
| (iv) | 23400 | 16800 | 19900 | 20033 |

*Too Numerous To Count (TNTC)

TABLE II
E. coli recovery

| Sample | Plate 1 | Plate 2 | Plate 3 | Average |
|---|---|---|---|---|
| (v) | 40 | 60 | 80 | 60 |
| (vi) | 0 | 0 | 100 | 33 |
| (vii) | 1980 | 2730 | 710 | 1807 |
| (viii) | 2000 | 600 | 400 | 1000 |

From the above-reported data, it is evident that both S. aureus and E. coli were captured via binding to the iron oxide in the sodium acetate buffer at pH 4.8. By contrast, a significantly smaller number of microorganisms appear to be in the supernatant (i.e., unbound to the iron oxide).

### EXAMPLE 2

An experiment was performed to determine whether Staphylococcus aureus (S. aureus) could be extracted from a pooled urine sample. The recovery of the microorganism from the urine was evaluated by examination of cultures prepared as described below.

The pH of pooled urine from healthy male and female donors was adjusted to pH 4.8 with 0.1 M acetate buffer having a pH of 4.8. A 1.0 ml quantity of pH-adjusted urine was pipetted into a 2.0 ml microcentrifuge tube containing 50 mg of ferrosferric oxide having an average particle size of approximately 1.4 microns. A 0.01 ml quantity of a S. aureas ATCC 25923 suspension at 1×10⁶ CFU/ml was added to the tube.

The tube containing the above-described mixture were rotated on a Nutator mixer for two hours at ambient temperature to promote binding of the iron oxide with the S. aureus microorganisms. A neodymium magnet was then placed at the sides of the tubes for 30 seconds.

The supernatant was then removed from the tubes with a pipette. The undiluted supernatant was applied to growth plates as described in more detail below.

The iron oxide/microorganism complex in the microtube was then washed twice with the above-mentioned sodium acetate buffer. The complex was then resuspended with 1 ml of 0.154M sodium chloride solution. The undiluted suspension was applied to growth plates as described in more detail below.

A 0.1 ml quantity of each of the above-mentioned supernatant and suspension were pipetted and spread onto each one of 3 different BBL™ blood agar plates (TSA II with 5% sheep's blood). The plates were incubated at 36° C. in ambient air for 24 hours. To determine the total recovery, the number of colonies were counted on each plate and multiplied by 10. The numbers of colonies calculated are reported in Table III.

TABLE III
S. aureus recovery

| Sample | Plate 1 | Plate 2 | Plate 3 | Average |
|---|---|---|---|---|
| Supernatant | 3440 | 4290 | 3630 | 3786 |
| Suspension | ≧10,000* | ≧10,000 | ≧10,000 | ≧10,000 |

* = Too numerous to count entire plate, so 1/4 of one plate counted, multiplied by 4, then by 10 to arrive at rough estimate for all plates.

From the above-reported data, it is evident that both S. aureus was captured via binding to the iron oxide in the sodium acetate buffer at pH 4.8. By contrast, a significantly smaller number of microorganisms appear to be in the supernatant (i.e., unbound to the iron oxide).

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents.

1. A method comprising:
(i) providing a sample containing at least one whole living organism that has not been lysed in a container;
(ii) creating a mixture comprising the sample and at least one magnetically-responsive particle;
(iii) providing the mixture with a pH of less than about 7.0, wherein alteration of the surface charge properties of the at least one magnetically-responsive particle occurs, thereby causing the at least one whole living organism to become non-specifically reversibly bound to the at least one magnetically-responsive particle to form a complex;
(iv) applying a magnetic field to the complex;
(v) removing the remainder of the sample which is not bound to the at least one magnetically-responsive particle from the container while the magnetic field is applied to the complex;
(vi) washing the complex; and
(vii) eluting the at least one whole living organism from the at least one magnetically-responsive particle comprising raising the pH to about 8.3-8.4.

2. The method of claim 1, further comprising (viii) reapplying a magnetic field to the eluted at least one magnetically-responsive particle thereby removing the at least one magnetically-responsive particle from the solution with the at least one whole living organism.

3. The method of claim 2, further comprising (ix) removing the at least one whole living organism from the container.

4. The method of claim 1, wherein in step (iii) the at least one whole living organism becomes bound to the at least one magnetically-responsive particle without precipitation.

5. The method of claim 1, wherein the at least one magnetically-responsive particle comprises an uncoated, untreated particle.

6. The method of claim 5, wherein the at least one magnetically-responsive particle comprises iron oxide, ferric hydroxide or ferrosoferric oxide.

7. The method of claim 1, wherein step (iii) comprises providing the mixture with a pH of about 4.5-5.5.

8. A method comprising:
(i) providing a sample containing at least one whole living organism that has not been lysed in a container;
(ii) creating a mixture comprising the sample and at least one magnetically-responsive particle;
(iii) providing the mixture with a pH of less than about 7.0, wherein alteration of the surface charge properties of the at least one magnetically-responsive particle occurs, thereby causing the at least one whole living organism to become non-specifically bound to the at least one magnetically-responsive particle to form a complex;
(iv) applying a magnetic field to the complex;
(v) removing the remainder of the mixture from the container while the magnetic field is applied to the complex;
(vi) eluting the at least one whole living organism from the at least one magnetically-responsive particle comprising raising the pH to about 8.3-8.4; and
(vii) reapplying a magnetic field to the eluted at least one magnetically-responsive particle thereby removing the at least one magnetically-responsive particle from the solution with the at least one whole living organism.

9. The method of claim 8, further comprising (viii) removing the at least one whole living organism from the container.

10. The method of claim 8, wherein in step (iii) the at least one whole living organism becomes bound to the at least one magnetically-responsive particle without precipitation.

11. The method of claim 8, wherein the at least one magnetically-responsive particle comprises an uncoated, untreated particle.

12. The method of claim 11, wherein the magnetically-responsive particle comprises iron oxide, ferric hydroxide or ferrosoferric oxide.

13. The method of claim 8, wherein step (iii) comprises providing the mixture with a pH of about 4.5-5.5

14. The method of claim 1 wherein said at least one whole living organism comprises a whole cell, bacterium, virus, or parasite and combinations thereof.

15. The method of claim 8 wherein said at least one whole living organism comprises a whole cell, bacterium, virus, or parasite and combinations thereof.
